(19) United States
(12) Patent Application Publication
(10) Pub. No.: US 2007/0259893 A1
(43) Pub. Date: Nov. 8, 2007

(54) Pyrazolopyrimidines

(75) Inventors: Olaf Gebauer, Leverkusen (DE); Ulrich Heinemann, Leichlingen (DE); Stefan Herrmann, Langenfeld (DE); Herbert Gayer, Monheim (DE); Stefan Hillebrand, Neuss (DE); Hans-Ludwig Elbe, Wuppertal (DE); Ronald Ebbert, Nurnberg (DE); Ulrike Wachendorff-Neumann, Neuwied (DE); Peter Dahmen, Neus (DE); Karl-Heinz Kuck, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: BAYER CROPSCIENCE AKTIENGESELLSCHAFT, Monheim (DE)

(21) Appl. No.: 10/581,946

(22) PCT Filed: Dec. 9, 2004
(86) PCT No.: PCT/EP04/13988

§ 371 Date: May 14, 2007

(30) Foreign Application Priority Data
Dec. 10, 2003 (DE) 10357567.7

(51) Int. Cl. C07D 487/02; A61K 31/519

(52) U.S. Cl. 514/259.3; 544/281

(57) ABSTRACT

Novel pyrazolopyrimidines of the formula 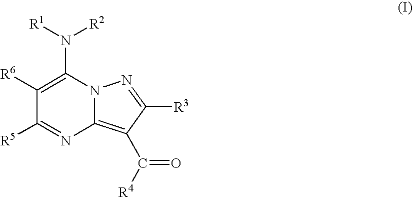 in which
R¹, R², R³, R⁴, R⁵ and R⁶ are as defined in the description, a plurality of processes for preparing these compounds and their use for controlling unwanted microorganisms.

The present invention relates to novel pyrazolopyrimidines, to a plurality of processes for preparing them and to their use for controlling unwanted microorganisms.

It is already known that certain pyrazolopyrimidines have fungicidal properties (compare DE-A 3 130 633 or FR-A 2 794 745).

However, since the ecological and economical demands made on modern fungicides are increasing constantly, for example with respect to activity spectrum, toxicity, selectivity, application rate, formation of residues and favorable manufacture, and there can furthermore be problems, for example, with resistance, there is a constant need to develop novel fungicides which, at least in some areas, have advantages over those of the prior art.

This invention now provides novel pyrazolopyrimidines of the formula 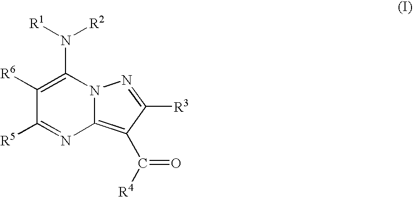
in which
R¹ represents optionally substituted alkyl, optionally substituted alkenyl, optionally substituted alkynyl, optionally substituted cycloalkyl or represents optionally substituted heterocyclyl,
R² represents hydrogen or alkyl, or
R¹ and R² together with the nitrogen atom to which they are attached represent an optionally substituted heterocyclic ring,
R³ represents hydrogen, halogen, optionally substituted alkyl or optionally substituted cycloalkyl,
R⁴ represents substituted alkyl, optionally substituted alkenyl, optionally substituted alkynyl, optionally substituted cycloalkyl or optionally substituted benzyl,
R⁵ represents halogen, optionally substituted alkyl, optionally substituted alkoxy, optionally substituted alkylthio, optionally substituted alkylsulfinyl or optionally substituted alkylsulfonyl and
R⁶ represents optionally substituted aryl.

Furthermore, it has been found that pyrazolopyrimidines of the formula (I) are obtained when
a) cyano compounds of the formula 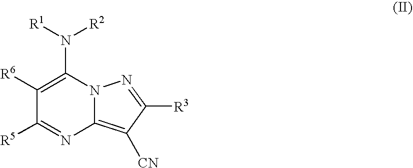
   in which
   R¹, R², R³, R⁵ and R⁶ are as defined above
   are reacted with Grignard compounds of the formula
      R⁷—Mg—X (III)
      in which
      R⁷ represents substituted alkyl, optionally substituted alkenyl, optionally substituted alkynyl, optionally substituted cycloalkyl or optionally substituted benzyl and
      X represents chlorine, bromine or iodine
      in the presence of a catalyst and in the presence of a diluent,
      or
b) pyrazolopyrimidines of the formula 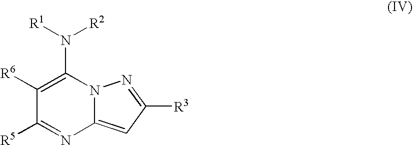
   in which
   R¹, R², R³, R⁵ and R⁶ are as defined above
   are reacted with acid halides of the formula 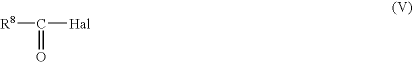
   in which
   R⁸ represents substituted alkyl, optionally substituted alkenyl, optionally substituted alkynyl, optionally substituted cycloalkyl or optionally substituted benzyl and
   Hal represents chlorine or bromine,
   or
   with acid anhydrides of the formula 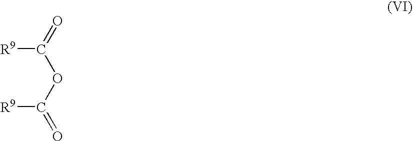
   in which
   R⁹ represents substituted alkyl or optionally substituted benzyl,
   or other activated carboxylic acid derivatives, such as 4-dimethylaminopyridine acid anhydride adducts,
   in each case in the presence of a catalyst and, if appropriate, in the presence of a diluent.

Finally, it has been found that the pyrazolopyrimidines of the formula (I) are highly suitable for controlling unwanted microorganisms. Especially, they have strong fungicidal activity and can be used both in crop protection and in the protection of materials.

Depending on the substitution pattern, the compounds according to the invention can, if appropriate, be present as mixtures of different possible isomeric forms, in particular of stereoisomers, such as E and Z, threo and erythro and also optical isomers, and, if appropriate, also in the form of tautomers. If R⁶ carries different substituents at the two atoms adjacent to the point of attachment, the compounds in question may be present in a particular stereoisomeric form, i.e. as atropisomers.

Formula (I) provides a general definition of the pyrazolopyrimidines according to the invention. Preference is given to those compounds of the formula (I) in which
R¹ represents alkyl having 1 to 6 carbon atoms which may be mono- to pentasubstituted by identical or different substituents from the group consisting of halogen, cyano, hydroxyl, alkoxy having 1 to 4 carbon atoms and cycloalkyl having 3 to 6 carbon atoms, or
R¹ represents alkenyl having 2 to 6 carbon atoms which may be mono- to trisubstituted by identical or different substituents from the group consisting of halogen, cyano, hydroxyl, alkoxy having 1 to 4 carbon atoms and cycloalkyl having 3 to 6 carbon atoms, or
R¹ represents alkynyl having 3 to 6 carbon atoms which may be mono- to trisubstituted by identical or different substituents from the group consisting of halogen, cyano, alkoxy having 1 to 4 carbon atoms and cycloalkyl having 3 to 6 carbon atoms, or
R¹ represents cycloalkyl having 3 to 6 carbon atoms which may be mono- to trisubstituted by identical or different substituents from the group consisting of halogen and alkyl having 1 to 4 carbon atoms, or
R¹ represents saturated or unsaturated heterocyclyl having 5 or 6 ring members and 1 to 3 heteroatoms, such as nitrogen, oxygen and/or sulfur, where the heterocyclyl may be mono- or disubstituted by halogen, alkyl having 1 to 4 carbon atoms, cyano, nitro and/or cycloalkyl having 3 to 6 carbon atoms,
R² represents hydrogen or alkyl having 1 to 4 carbon atoms, or
R¹ and R² together with the nitrogen atom to which they are attached represent a saturated or unsaturated heterocyclic ring having 3 to 6 ring members, where the heterocycle may contain a further nitrogen, oxygen or sulfur atom as ring member and where the heterocycle may be substituted up to three times by fluorine, chlorine, bromine, alkyl having 1 to 4 carbon atoms and/or haloalkyl having 1 to 4 carbon atoms and 1 to 9 fluorine and/or chlorine atoms,
R³ represents hydrogen, fluorine, chlorine, bromine, iodine, alkyl having 1 to 4 carbon atoms, haloalkyl having 1 to 4 carbon atoms and 1 to 9 halogen atoms or represents cycloalkyl having 3 to 6 carbon atoms,
R⁴ represents haloalkyl having 1 to 6 carbon atoms, alkenyl having 2 to 6 carbon atoms, alkynyl having 2 to 6 carbon atoms, cycloalkyl having 3 to 6 carbon atoms or represents benzyl,
R⁵ represents fluorine, chlorine, bromine, alkyl having 1 to 4 carbon atoms, alkoxy having 1 to 4 carbon atoms, alkylthio having 1 to 4 carbon atoms, alkylsulfinyl having 1 to 4 carbon atoms or alkylsulfonyl having 1 to 4 carbon atoms, and
R⁶ represents phenyl which may be mono- to tetrasubstituted by identical or different substituents from the group consisting of halogen, cyano, nitro, amino, hydroxyl, formyl, carboxyl, carbamoyl, thiocarbamoyl;
   in each case straight-chain or branched alkyl, alkoxy, alkylthio, alkylsulfinyl or alkylsulfonyl having in each case 1 to 6 carbon atoms;
   in each case straight-chain or branched haloalkyl, haloalkoxy, haloalkylthio, haloalkylsulfinyl or haloalkylsulfonyl having in each case 1 to 6 carbon atoms and 1 to 13 identical or different halogen atoms;
   in each case straight-chain or branched haloalkenyl or haloalkenyloxy having in each case 2 to 6 carbon atoms and 1 to 11 identical or different halogen atoms;
   in each case straight-chain or branched alkylamino, dialkylamino, alkylcarbonyl, alkylcarbonyloxy, alkoxycarbonyl, alkylsulfonyloxy, hydroximinoalkyl or alkoximinoalkyl having in each case 1 to 6 carbon atoms in the individual alkyl moieties;
   cycloalkyl having 3 to 6 carbon atoms,
   2,3-attached 1,3-propanediyl, 1,4-butanediyl, methylenedioxy (—O—CH₂—O—) or 1,2-ethylenedioxy (—O—CH₂—CH₂—O—), where these radicals may be mono- or polysubstituted by identical or different substituents from the group consisting of halogen, alkyl having 1 to 4 carbon atoms and haloalkyl having 1 to 4 carbon atoms and 1 to 9 identical or different halogen atoms.

Particular preference is given to those pyrazolopyrimidines of the formula (I), in which
R¹ represents a radical of the formula 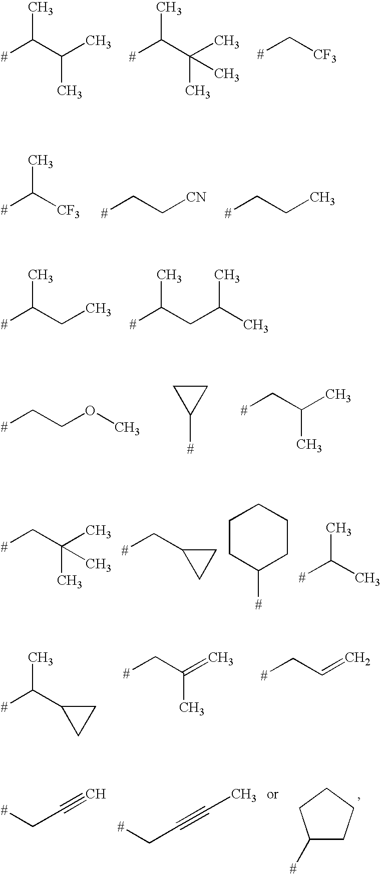
where # denotes the point of attachment and where, for the radicals which may be present in optically active form, each of the possible stereoisomers or mixtures thereof may be present,
R² represents hydrogen, methyl, ethyl or propyl, or
R¹ and R² together with the nitrogen atom to which they are attached represent pyrrolidinyl, piperidinyl, morpholinyl, thiomorpholinyl, piperazinyl, 3,6-dihydro-1(2H)-piperidinyl or tetrahydro-1(2H)-pyridazinyl, where these radicals may be substituted by 1 to 3 fluorine atoms, 1 to 3 methyl groups and/or trifluoromethyl,
or
R¹ and R² together with the nitrogen atom to which they are attached represent a radical of the formula 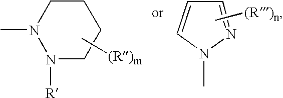
in which
R′ represents hydrogen or methyl,
R″ represents methyl, ethyl, fluorine, chlorine or trifluoromethyl,
m represents the number 0, 1, 2 or 3, where R″ represents identical or different radicals, if m represents 2 or 3,
R′″ represents methyl, ethyl, fluorine, chlorine or trifluoromethyl
and
n represents the number 0, 1, 2 or 3, where R′″ represents identical or different radicals if n represents 2 or 3,
R³ represents hydrogen, fluorine, chlorine, bromine, iodine, methyl, ethyl, isopropyl, cyclopropyl, cyclobutyl, cyclopentyl, cyclohexyl, trifluoromethyl, 1-trifluoromethyl-2,2,2-trifluoroethyl or heptafluoroisopropyl,
R⁴ represents haloalkyl having 1 to 4 carbon atoms, alkenyl having 3 to 5 carbon atoms, alkynyl having 3 to 5 carbon atoms, cyclopropyl, cyclopentyl, cyclohexyl or represents benzyl,
R⁵ represents fluorine, chlorine, bromine, methyl, ethyl, methoxy, ethoxy, methylthio, methylsulfinyl or methylsulfonyl, and
R⁶ represents phenyl which may be mono- to trisubstituted by identical or different substituents from the group consisting of fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl; methoximinoethyl, ethoximinoethyl, cyclopropyl, cyclobutyl, cyclopentyl or cyclohexyl,
   2,3-attached 1,3-propanediyl, methylenedioxy (—O—CH₂—O—) or 1,2-ethylenedioxy (—O—CH₂—CH₂—O—), where these radicals may be mono- or polysubstituted by identical or different substituents from the group consisting of fluorine, chlorine, methyl, ethyl, n-propyl, i-propyl and trifluoromethyl.

A very particularly preferred group of compounds according to the invention are pyrazolopyrimidines of the formula (I), in which
R¹, R² and R³ have the particularly preferred meanings given above;
R⁴ represents CF₃, CCl₃, allyl, propargyl, cyclopropyl or benzyl,
R⁵ represents fluorine, chlorine, bromine, methyl, methoxy or methylthio and
R⁶ represents 2,4-, 2,5- or 2,6-disubstituted phenyl, or 2-substituted phenyl or represents 2,4,6-trisubstituted phenyl, possible substituents being the radicals which have been mentioned in the context of the enumeration of the particularly preferred definitions.

The radical definitions mentioned above can be combined with one another as desired. Moreover, individual definitions may not apply.

Using 3-cyano-5-chloro-6-(2-chloro-4-fluorophenyl)-7-(4-methylpiperidino)pyrazolo[1,5-a]pyrimidine as starting material and benzylmagnesium bromide as reaction component, the course of the process (a) according to the invention can be illustrated by the formula scheme below.

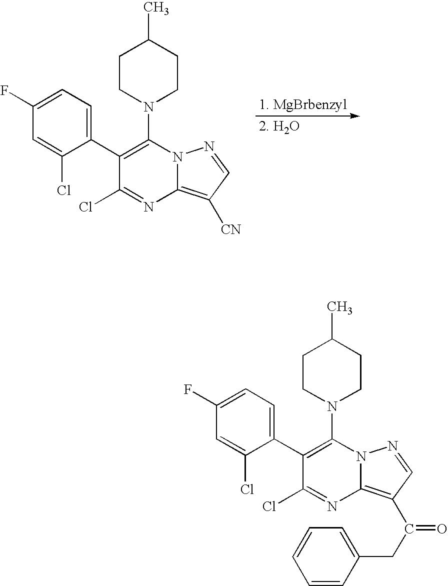

Using 5-chloro-6-(2-chlorophenyl)-7-(1,2-dimethylpropylamino)pyrazolo[1,5-a]pyrimidine as starting material, 4-dimethylamino-1-trifluoroacetylpyridiniumtrifluoroacetate as reaction component and aluminum trichloride as catalyst, the course of the process (b) according to the invention can be illustrated by the formula scheme below.

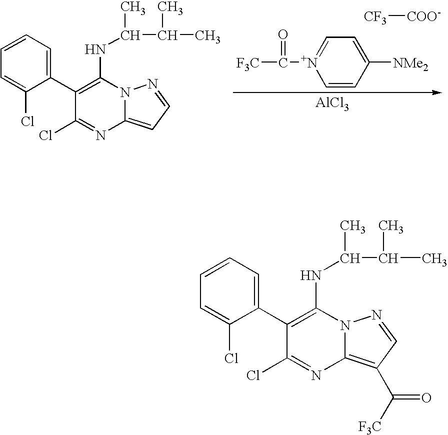

The formula (II) provides a general definition of the cyano compounds required as starting materials for carrying out the process (a) according to the invention. In this formula, R¹, R², R³, R⁵ and R⁶ preferably have those meanings which have already been mentioned in connection with the description of the compounds of the formula (I) according to the invention as being preferred for these radicals.

The cyano compounds of the formula (II) can be prepared by
d) reacting halopyrazolopyrimidines of the formula 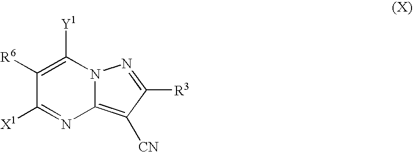
   in which
   R³ and R⁶ are as defined above,
   X¹ represents halogen and
   Y¹ represents halogen,
   with amines of the formula 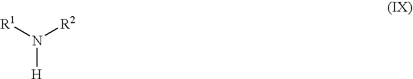
   in which
   R¹ and R² are as defined above,
   if appropriate in the presence of a diluent, if appropriate in the presence of a catalyst and if appropriate in the presence of an acid acceptor,
   and, if appropriate, the resulting cyano compounds of the formula 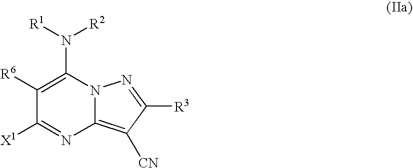
   in which
   R¹, R², R³, R⁶ and X¹ are as defined above
   are, in a second step, reacted with compounds of the formula
      R¹⁰-Me (XI)
   R¹⁰ represents optionally substituted alkoxy, optionally substituted alkylthio, optionally substituted alkylsulfinyl or optionally substituted alkylsulfonyl and
   Me represents sodium or potassium,
   if appropriate in the presence of a diluent.

The halopyrazolopyrimidines of the formula (X) are known or can be prepared by known methods (cf. DE-A 103 28 996 and PCT/EP 03/05 159).

Thus, halopyrazolopyrimidines of the formula (X) are obtained when
e) dihydroxypyrazolopyrimidines of the formula 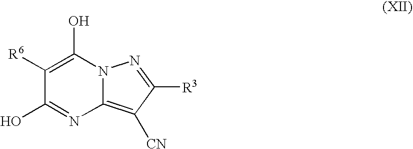
   in which
   R³ and R⁶ are as defined above
   are reacted with halogenating agents, if appropriate in the presence of a diluent.

The dihydroxypyrazolopyrimidines of the formula (XII) can be prepared by
f) reacting arylmalonic esters of the formula 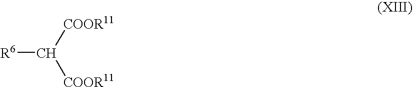
   in which
   R⁶ is as defined above and
   R¹¹ represents alkyl
   with aminopyrazoles of the formula 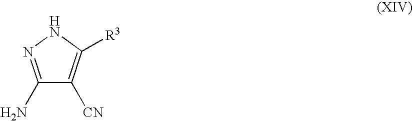
   in which
   R³ is as defined above,
   if appropriate in the presence of a diluent and if appropriate in the presence of a strong base.

The formula (XIII) provides a general definition of the arylmalonic esters required as starting materials for carrying out the process (f). In this formula, R⁶ preferably has those meanings which have already been mentioned in connection with the description of the compounds of the formula (I) according to the invention as being preferred for this radical. R¹¹ preferably represents alkyl having 1 to 4 carbon atoms, particularly preferably methyl or ethyl.

The arylmalonic esters of the formula (XIII) are known or can be prepared by known methods (cf. U.S. Pat. No. 6,156,925).

The aminopyrazoles of the formula (XIV) are likewise known or can be prepared by known methods.

Suitable diluents for carrying out the process (f) are all customary inert organic solvents. Preference is given to using aliphatic, alicyclic or aromatic hydrocarbons, such as petroleum ether, hexane, heptane, cyclohexane, methylcyclohexane, benzene, toluene, xylene or decalin; halogenated hydrocarbons, such as chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methyl-formanilide, N-methylpyrrolidone or hexamethylphosphoric triamide; esters, such as methyl acetate or ethyl acetate; sulfoxides, such as dimethyl sulfoxide; sulfones, such as sulfolane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether; amines, such as tri-n-butylamine or carboxylic acids, such as acetic acid. Suitable strong bases for carrying out the process (f) are, preferably, alkaline earth metal or alkali metal hydrides or alkoxides, and also alkali metal amides. Sodium hydride, sodium amide, sodium methoxide, sodium ethoxide and potassium tert-butoxide may be mentioned by way of example.

Both the process (f) and the other processes described in the present patent application are generally carried out under atmospheric pressure. However, it is also possible to operate under elevated pressure or—as long as no highly volatile reaction components are present—under reduced pressure.

When carrying out the process (f), the reaction temperatures can in each case be varied within a relatively wide range. In the absence of bases, the process is generally carried out at temperatures between 100° C. and 250° C., preferably between 120° C. and 200° C. In the presence of bases, the process is generally carried out at temperatures between 20° C. and 120° C., preferably between 20° C. and 80° C.

When carrying out the process (f), in general from 1 to 15 mol, preferably from 1 to 8 mol, of aminopyrazole of the formula (XIV) are employed per mole of arylmalonic ester of the formula (XIII). Work-up is carried out by customary methods.

Suitable halogenating agents for carrying out the process (e) according to the invention are all customary reagents suitable for exchanging hydroxyl groups attached to carbon for halogen. Preference is given to using phosphorus trichloride, phosphorus tribromide, phosphorus pentachloride, phosphorus oxychloride, phosgene, thionyl chloride, thionyl bromide or mixtures thereof. The corresponding fluorine compounds of the formula (VI) can be prepared from the chlorine or bromine compounds by reaction with potassium fluoride.

Suitable diluents for carrying out the process (e) are all organic solvents customary for such halbgenations. Preference is given to using aliphatic, alicyclic or aromatic hydrocarbons, such as petroleum ether, hexane, heptane, cyclohexane, methylcyclohexane, benzene, toluene, xylene or decalin; halogenated hydrocarbons, such as chlorobenzene, dichlorobenzene, dichloromethane, chloroform, carbon tetrachloride, dichloroethane or trichloroethane.

However, it is also possible for the halogenating agent itself or a mixture of halogenating agent and one of the diluents mentioned to serve as diluent.

When carrying out the process (e), the reaction temperatures can in each case be varied within a relatively wide range. In general, the process is carried out at temperatures between 20° C. and 150° C., preferably between 40° C. and 120° C.

When carrying out the process (e), in each case an excess of halogenating agent is employed per mole of dihydroxypyrazolopyrimidine of the formula (XII). Work-up is carried out by customary methods.

The formula (X) provides a general definition of the halopyrazolopyrimidines required as starting materials for carrying out the process (d). In this formula, R³ and R⁶ preferably have those meanings which have already been mentioned in connection with the description of the compounds of the formula (I) according to the invention as being preferred for these radicals. X¹ and Y¹ each preferably represent fluorine, chlorine or bromine, particularly preferably fluorine or chlorine.

The formula (IX) provides a general definition of the amines required as reaction components for carrying out the process (c) and also the process (d). In this formula, R¹ and R² preferably have those meanings which have already been mentioned in connection with the description of the compounds of the formula (I) according to the invention as being preferred for these radicals.

The formula (XI) provides a general definition of the compounds required as reaction components in the second step of process (d). In this formula, R¹⁰ preferably represents alkoxy having 1 to 4 carbon atoms, alkylthio having 1 to 4 carbon atoms, alkylsulfinyl having 1 to 4 carbon atoms or alkylsulfonyl having 1 to 4 carbon atoms. Me also preferably represents sodium or potassium.

Particular preference is given to compounds of the formula (XI) in which R¹⁰ represents methoxy, ethoxy, methylthio, methylsulfinyl or methylsulfonyl and Me represents sodium or potassium.

The amines of the formula (IX) and also the compounds of the formula (XI) are known or can be prepared by known methods.

Suitable diluents for carrying out the first step of the process (d) are all customary inert organic solvents. Preference is given to using halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters such as methyl acetate or ethyl acetate; sulfoxides, such as dimethyl sulfoxide; sulfones, such as sulfonane.

Suitable acid acceptors for carrying out the first step of the process (d) are all inorganic or organic bases customary for such reactions. Preference is given to using alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, potassium acetate, calcium acetate, sodium carbonate, potassium carbonate, potassium bicarbonate and sodium bicarbonate, and furthermore ammonium compounds, such as ammonium hydroxide, ammonium acetate and ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

Suitable catalysts for carrying out the first step of the process (d) are all reaction promoters customary for such reactions. Preference is given to using fluorides, such as sodium fluoride, potassium fluoride or ammonium fluoride.

When carrying out the first step of the process (d), the reaction temperatures can be varied within a relatively wide range. In general, the process is carried out at temperatures between 0° C. and 150° C., preferably at temperatures between 0° C. and 80° C.

When carrying out the first step of the process (d), in general from 0.5 to 10 mol, preferably from 0.8 to 2 mol, of amine of the formula (IX) are employed per mole of halopyrazolopyrimidine of the formula (X). Work-up is carried out by customary methods.

Suitable diluents for carrying out the second step of the process (d) are all customary inert organic solvents. Preference is given to using halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulfoxides, such as dimethyl sulfoxide; sulfones, such as sulfolane.

When carrying out the second step of the process (d), the reaction temperatures may also be varied within a relatively wide range. In general, the process is carried out at temperatures between 0° C. and 150° C., preferably between 20° C. and 100° C.

When carrying out the second step of the process (d), the cyano compound of the formula (IIa) in question is reacted with an equivalent amount or an excess of a compound of the formula (XI). Work-up is carried out by customary methods.

The formula (III) provides a general definition of the Grignard compounds required as reaction components for carrying out the process (a) according to the invention. In this formula
R⁷ preferably represents substituted alkyl having 1 to 6 carbon atoms, alkenyl having 2 to 6 carbon atoms, alkynyl having 2 to 6 carbon atoms, cycloalkyl having 3 to 6 carbon atoms or benzyl,
X also preferably represents chlorine, bromine or iodine.

Particular preference is given to Grignard compounds of the formula (III) in which
R⁷ represents substituted alkyl having 1 to 4 carbon atoms, alkynyl having 3 to 5 carbon atoms, cyclopropyl, cyclopentyl, cyclohexyl or benzyl and
X represents chlorine, bromine or iodine.

Very particular preference is given to compounds of the formula (III) in which
R⁷ represents allyl, propargyl, cyclopropyl or benzyl and
X represents chlorine, bromine or iodine.

The Grignard compounds of the formula (III) are known or can be prepared by known methods.

Suitable catalysts for carrying out the process (a) according to the invention are all reaction promoters customary for Grignard reactions. Potassium iodide and iodine may be mentioned by way of example.

Suitable diluents for carrying out the process (a, variant β) according to the invention are all inert organic solvents customary for such reactions. Preference is given to using ethers, such as diethyl ether, dioxane or tetrahydrofuran, furthermore aromatic hydrocarbons, such as toluene, and also mixtures of ethers and aromatic hydrocarbons, such as toluene/tetrahydrofuran.

When carrying out the process (a) according to the invention, the reaction temperatures can be varied within a relatively wide range. In general, the process is carried out at temperatures between −20° C. and +100° C., preferably between 0° C. and 80° C.

When carrying out the process (a) according to the invention, in general from 2 to 3 mol of Grignard compound of the formula (III) are employed per mole of cyano compound of the formula (II). This is followed by aqueous work-up according to customary methods.

The formula (IV) provides a general definition of the pyrazolopyrimidines required as starting materials for carrying out the process (b) according to the invention. In this formula, R¹, R², R³, R⁵ and R⁶ preferably have those meanings which have already been mentioned in connection with the description of the compounds of the formula (I) according to the invention as being preferred for these radicals.

The pyrazolopyrimidines of the formula (IV) are known (see, for example, U.S. Pat. No. 6,552,026), or they can be prepared by known methods.

The formulae (V) and (VI) provide general definitions of the acid halides and acid anhydrides required as reaction components for carrying out the process (b) according to the invention. In the formula (V),
R⁸ preferably represents alkyl having 1 to 6 carbon atoms, alkenyl having 2 to 6 carbon atoms, alkynyl having 2 to 6 carbon atoms, cycloalkyl having 3 to 6 carbon atoms or benzyl, and
Hal preferably represents chlorine or bromine.

Particular preference is given to acid halides of the formula (V) in which
R⁸ represents substituted alkyl having 1 to 4 carbon atoms, alkenyl having 3 to 5 carbon atoms, alkynyl having 3 to 5 carbon atoms, cyclopropyl, cyclopentyl, cyclohexyl or benzyl and
Hal represents chlorine or bromine.

Very particular preference is given to acid halides of the formula (V) in which
R⁸ represents allyl, propargyl, cyclopropyl or benzyl and
Hal represents chlorine or bromine.

In the formula (VI),
R⁹ preferably represents benzyl.

Particular preference is given to acid anhydrides of the formula (VI) in which

A preferred 4-dimethylaminopyridine acid anhydride adduct is, for example, the commercially available 4-dimethylamino-1-trifluoroacetylpyridinium trifluoracetate, the preparation of which is described in Synthesis 1996 (9), 1093.

Suitable catalysts for carrying out the process (b) according to the invention are all reaction promoters customarily used for Friedel-Crafts reactions. Preference is given to using Lewis acids, such as aluminum trichloride, aluminum tribromide and iron(III) chloride.

Suitable diluents for carrying out the process (b) according to the invention are all customary inert organic solvents which can be used for Friedel-Crafts reactions; preference is given to using ethers, such as diethyl ether, methyl tert-butyl ether, dioxane or tetrahydrofuran, and also carbon disulfide.

When carrying out the process (b) according to the invention, the reaction temperatures can be varied within a certain range. In general, the process is carried out at temperatures between −10° C. and +100° C., preferably between 0° C. and 80° C.

When carrying out the process (b) according to the invention, in general from 1 to 5 mol, preferably from 1 to 2 mol of acid halide of the formula (V) and from 1.1 to 5 mol, preferably from 1.1 to 3 mol, of catalyst, or from 1 to 5 mol, preferably from 1 to 2 mol of acid anhydride of the formula (VI) and from 2.1 to 6 mol, preferably from 2.1 to 4 mol, of catalyst are employed per mole of pyrazolopyrimidine of the formula (IV). In general, the reaction components are initially mixed at low temperature and, after the initially vigorous reaction has subsided, slowly heated to reflux temperature. Work-up is carried out by customary methods. The formula (VII) provides a general definition of the hydroxypyrazolopyrimidines required as starting materials for carrying out the process (e) according to the invention. In this formula, R³ and R⁶ preferably have those meanings which have already been mentioned in connection with the description of the compounds of the formula (I) according to the invention being preferred for these radicals.

The hydroxylpyrazolopyrimidines of the formula (VII) can be prepared according to the process (f) when aminopyrazoles of the formula (XIV) are employed which, instead of the CN group, carry a hydrogen atom.

The compounds according to the invention have potent microbicidal activity and can be employed for controlling unwanted microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora;

Pythium species, such as, for example, Pythium ultimum;

Phytophthora species, such as, for example, Phytophthora infestans;

Pseudoperonospora species, such as, for example, Pseudoperonospora humuli or

Pseudoperonospora cubensis;

Plasmopara species, such as, for example, Plasmopara viticola;

Bremia species, such as, for example, Bremia lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturia inaequalis;

Pyrenophora species, such as, for example, Pyrenophora teres or P. graminea

(conidia form: Drechslera, syn: Helminthosporium);

Cochliobolus species, such as, for example, Cochliobolus sativus

(conidia form: Drechslera, syn: Helminthosporium);

Uromyces species, such as, for example, Uromyces appendiculatus;

Puccinia species, such as, for example, Puccinia recondita;

Sclerotinia species, such as, for example, Sclerotinia scierotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as, for example, Septoria nodorum;

Leptosphaeria species, such as, for example, Leptosphaeria nodorum;

Cercospora species, such as, for example, Cercospora canescens;

Alternaria species, such as, for example, Alternaria brassicae; and

Pseudocercosporella species, such as, for example, Pseudocercosporella herpotrichoides.

The active compounds according to the invention also show a strong invigorating action in plants. Accordingly, they are suitable for mobilizing the internal defenses of the plant against attack by unwanted microorganisms.

In the present context, plant-invigorating (resistance-inducing) compounds are to be understood as meaning substances which are capable of stimulating the defense system of plants such that, when the treated plants are subsequently inoculated with unwanted microorganisms, they display substantial resistance to these microorganisms.

In the present case, unwanted microorganisms are to be understood as meaning phytopathogenic fungi, bacteria and viruses. The compounds according to the invention can thus be used to protect plants within a certain period of time after treatment against attack by the pathogens mentioned. The period of time for which this protection is achieved generally extends for 1 to 10 days, preferably 1 to 7 days, from the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be employed with particularly good results for controlling cereal diseases, such as, for example, against Erysiphe species, and of diseases in viticulture and in the cultivation of fruit and vegetables, such as, for example, against Botrytis, Venturia, Sphaerotheca and Podosphaera species.

The active compounds according to the invention are also suitable for increasing the yield of crops. In addition, they show reduced toxicity and are well tolerated by plants.

If appropriate, the active compounds according to the invention can, at certain concentrations and application rates, also be employed as herbicides, for regulating plant growth and for controlling animal pests. If appropriate, they can also be used as intermediates or precursors in the synthesis of other active compounds.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multilayer coating.

In the protection of materials, the compounds according to the invention can be employed for protecting industrial materials against infection with, and destruction by, unwanted microorganisms.

Industrial materials in the present context are understood as meaning non-living materials which have been prepared for use in industry. For example, industrial materials which are intended to be protected by active compounds according to the invention from microbial change or destruction can be tackifiers, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular molds, wood-discolouring and wood-destroying fungi (Basidiomycetes) and against slime organisms and algae.

Microorganisms of the following genera may be mentioned as examples:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa, and

Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulfoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, pumice, marble, sepiolite and dolomite, or else synthetic granules of inorganic and organic meals, and granules of organic material such as sawdust, coconut shells, corn cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates, or else protein hydrolysates. Suitable dispersants are: for example lignosulfite waste liquors and methylcellulose.

Tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are, for example, the following compounds:

Fungicides:

2-phenylphenol; 8-hydroxyquinoline sulfate; acibenzolar-5-methyl; aldimorph; amidoflumet; ampropylfos; ampropylfos-potassium; andoprim; anilazine; azaconazole; azoxystrobin; benalaxyl; benalaxyl-M, benodanil; benomyl; benthiavalicarb-isopropyl; benzamacril; benzamacril-isobutyl; bilanafos; binapacryl; biphenyl; bitertanol; blasticidin-S; boscalid; bromuconazole; bupirimate; buthiobate; butylamine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichione; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)—N-[2-[4-[[3-(4-chlorophenyl)-2-propynyl]oxy]-3-methoxyphenyl]ethyl]-3-methyl-2-[(methylsulfonyl)amino]-butanamide; 1-(1-naphthalenyl)-1H-pyrrole-2,5-dione; 2,3,5,6-tetrachloro-4-(methylsulfonyl)-pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2, 2-dimethyl-1H-inden-1-yl)-1H-imidazole-5-carboxylate; monopotassium carbonate; N-(6-methoxy-3-pyridinyl)-cyclopropanecarboxamide; N-butyl-8-(1,1-dimethylethyl)-1-oxa-spiro[4,5]decane-3-amine; sodium tetracarbonate;

and copper salts and preparations, such as Bordeaux mixture; copper hydroxide; copper naphthenate; copper oxychloride; copper sulfate; cufraneb; copper oxide; mancopper; oxine-copper.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Insecticides/Acaricides/Nematicides:

1. Acetylcholinesterase (AChE) inhibitors

1.1 carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)

1.2 organophosphates (for example acephate, azamethiphos, azinphos (-methyl, -ethyl), bromophos-ethyl, bromfenvinfos (-methyl), butathiofos, cadusafos, carbophenothion, chloroethoxyfos, chlorofenvinphos, chloromephos, chloropyrifos (-methyl/-ethyl), coumaphos, cyanofenphos, cyanophos, chlorofenvinphos, demeton-S-methyl, demeton-S-methylsulfone, dialifos, diazinon, dichlofenthion, dichlorovos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl o-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathiuon, quinalphos, sebufos, sulfotep, sulprofos, tebupirimfos, temephos, terbufos, tetrachlorvinphos, thiometon, triazophos, triclorfon, vamidothion)

2. Sodium Channel Modulators/Blockers of Voltage-Gated Sodium Channels

2.1 pyrethroids (for example acrinathrin, allethrin (d-cis-trans, d-trans), beta-cyfluthrin, bifenthrin, bioallethrin, bioallethrin-S-cyclopentyl-isomer, bioethanomethrin, biopermethrin, bioresmethrin, chlovaporthrin, cis-cypermethrin, cis-resmethrin, cis-pernethrin, clocythrin, cycloprothrin, cyfluthrin, cyhalothrin, cypermethrin (alpha-, beta-, theta-, zeta-), cyphenothrin, DDT, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R-isomer), tralomethrin, transfluthrin, ZXI 8901, pyrethrins (pyrethrum))

2.2 oxadiazines (for example indoxacarb)

3. Acetylcholine Receptor Agonists/Antagonists

3.1 chloronicotinyls/neonicotinoids (for example acetamiprid, clothianidin, dinotefuran, imidacloprid, nitenpyram, nithiazine, thiacloprid, thiamethoxam)

3.2 nicotine, bensultap, cartap

4. Acetylcholine Receptor Modulators

4.1 spinosyns (for example spinosad)

5. Antagonists of GABA-Gated Chloride Channels

5.1 cyclodiene organochlorines (for example camphechlor, chlordane, endosulfan, gamma-HCH, HCH, heptachlor, lindane, methoxychlor

5.2 fiproles (for example acetoprole, ethiprole, fipronil, vaniliprole)

6. Chloride Channel Activators

6.1 mectins (for example abamectin, avermectin, emamectin, emamectin-benzoate, ivermectin, milbemectin, milbemycin)

7. Juvenile Hormone Mimetics

(for example diofenolan, epofenonane, fenoxycarb, hydroprene, kinoprene, methoprene, pyriproxifen, triprene)

8. Ecdyson agonists/disruptors

8.1 diacylhydrazines (for example chromafenozide, halofenozide, methoxyfenozide, tebufenozide)

9. Chitin Biosynthesis Inhibitors

9.1 benzoylureas (for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron)

9.2 buprofezin

9.3 cyromazine

10. Inhibitors of Oxidative Phosphorylation, ATP Disruptors

10.1 diafenthiuron

10.2 organotins (for example azocyclotin, cyhexatin, fenbutatin-oxide)

11. Decouplers of Oxidative Phosphorylation Acting by Interrupting the H-Proton Gradient

11.1 pyrroles (for example chlorfenapyr)

11.2 dinitrophenols (for example binapacryl, dinobuton, dinocap, DNOC)

12. Site-I Electron Transport Inhibitors

12.1 METIs (for example fenazaquin, fenpyroximate, pyrimidifen, pyridaben, tebufenpyrad, tolfenpyrad)

12.2 hydramethylnone

12.3 dicofol

13. Site-II Electron Transport Inhibitors

13.1 rotenone

14. Site-III Electron Transport Inhibitors

14.1 acequinocyl, fluacrypyrim

15. Microbial Disruptors of the Insect Gut Membrane

Bacillus thuringiensis strains

16. Inhibitors of Fat Synthesis

16.1 tetronic acids (for example spirodiclofen, spiromesifen)

16.2 tetramic acids [for example 3-(2,5-dimethylphenyl)-8-methoxy-2-oxo-1-azaspiro[4,5]dec-3-en-4-yl ethyl carbonate (alias: carbonic acid, 3-(2,5-dimethylphenyl)-8-methoxy-2-oxo-1-azaspiro[4,5]dec-3-en-4-yl ethyl ester, CAS Reg. No.: 382608-10-8) and carbonic acid, cis-3-(2,5-dimethylphenyl)-8-methoxy-2-oxo-1-azaspiro[4,5]dec-3-enyl ethyl ester (CAS Reg. No.: 203313-25-1)]

17. Carboxamides

(for example flonicamid)

18. Octopaminergic Agonists

(for example amitraz)

19. Inhibitors of Magnesium-Stimulated ATPase

(for example propargite)

20. Phthalamides

(for example N²-[1,1-dimethyl-2-(methylsulfonyl)ethyl]-3-iodo-N′-[2-methyl-4-[1,2,2,2-tetrafluoro-1-(trifluoromethyl)ethyl]phenyl]-1,2-benzenedicarboxamide (CAS Reg. No.: 272451-65-7), flubendiamide)

21. Nereistoxin Analogues

(for example thiocyclam hydrogen oxalate, thiosultap-sodium)

22. Biologicals, Hormones or Pheromones

(for example azadirachtin, Bacillus spec., Beauveria spec., codlemone, Metarrhizium spec., Paecilomyces spec., thuringiensin, Verticillium spec.)

23. Active Compounds with Unknown or Unspecific Mechanisms of Action

23.1 fumigants (for example aluminium phosphide, methyl bromide, sulfuryl fluoride)

23.2 selective antifeedants (for example cryolite, flonicamid, pymetrozine)

23.3 mite growth inhibitors (for example clofentezine, etoxazole, hexythiazox)

23.4 amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin, furthermore the compound 3-methylphenyl propylcarbamate (Tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo-isomer (CAS Reg. No. 185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

In addition, the compounds of the formula (I) according to the invention also have very good antimycotic activity. They have, a very broad antimycotic activity spectrum in particular against dermatophytes and yeasts, molds and diphasic fungi (for example against Candida species such as Candida albicans, Candida glabrata) and Epidemmophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does by no means limit the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10 000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10 000 g/ha, preferably between 1 and 5000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), corn, soybeans, potatoes, cotton, tobacco, oilseed rape and also fruit plants (with the fruits apples, pears, citrus fruits and grapes), and particular emphasis is given to corn, soybeans, potatoes, cotton, tobacco and oilseed rape. Traits that are particularly emphasized are increased defense of the plants against insects, arachnids, nematodes and slugs and snails by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defense of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulfonylureas, glyphosate or phosphinotricin (for example the “PAT” gene). The genes which impart the desired traits in question can also be present in combination with one another in the transgenic plants. Examples of “Bt plants” which may be mentioned are corn varieties, cotton varieties, soybean varieties and potato varieties which are sold under the trade names YIELD GARDE (for example corn, cotton, soybeans), KnockOut® (for example com), —-StarLink® (for example corn), Bollgard® (cotton), Nucoton® (cotton) and NewLeaf® (potato). Examples of herbicide-tolerant plants which may be mentioned are corn varieties, cotton varieties and soybean varieties which are sold under the trade names Roundup Ready® (tolerance to glyphosate, for example corn, cotton, soybean), Liberty Link® (tolerance to phosphinotricin, for example oilseed rape), IMI® (tolerance to imidazolinones) and STS® (tolerance to sulfonylureas, for example corn). Herbicide-resistant plants (plants bred in a conventional manner for herbicide tolerance) which may be mentioned also include the varieties sold under the name Clearfield® (for example corn). Of course, these statements also apply to plant cultivars which have these genetic traits or genetic traits still to be developed, and which will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The compounds of the formula (I) according to the invention are furthermore suitable for suppressing the growth of tumour cells in humans and mammals. This is based on an interaction of the compounds according to the invention with tubulin and microtubuli and by promoting microtubuli polymerization.

For this purpose, it is possible to administer an effective amount of one or more compounds of the formula (I) or pharmaceutically acceptable salts thereof.

The preparation and the use of the active compounds according to the invention is illustrated in the examples below.

### PREPARATION EXAMPLES

### Example 1

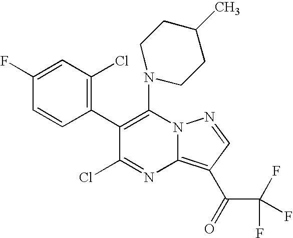

0.500 g (1.318 mmol) of 5-chloro-6-(2-chloro-4-fluorophenyl)-7-(4-methylpiperidino)-pyrazolo[1,5-a]pyrimidine and 0.438 g (1.318 mmol) of 4-dimethylamino-1-trifluoroacetyl-pyridinium trifluoracetate are initially charged in 66.0 g of 1,2-dichlorethane and cooled to 0° C., and 0.439 g (3.296 mmol) of aluminum trichloride is added, the temperature not exceeding 5° C. The mixture was stirred at room temperature overnight. The mixture was concentrated, a mixture of ethyl acetate and water was added and the mixture was acidified with hydrochloric acid. After extraction, the organic phase was dried and concentrated.

This gave 3-trifluoroacetyl-5-chloro-6-(2-chloro-4-fluorophenyl)-7-(4-methylpiperidino)-pyrazolo[1,5-a]pyrimidine.

HPLC: logP=5.44

### Example 2

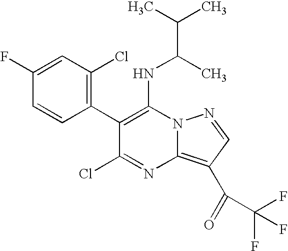

HPLC: logP=5.08

was obtained analogously

Preparation of Starting Materials

### Example 3

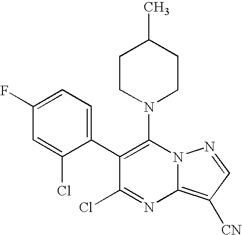 Process (d):

At room temperature, a solution of 5 mmol of 3-cyano-5,7-dichloro-6-(2-chloro-4-fluorophenyl)-pyrazolo[1,5-a]pyrimidine in 10 ml of acetonitrile is added dropwise with stirring to a mixture of 30 ml of acetonitrile, 5 mmol of potassium carbonate and 5 mmol of 4-methylpiperidine. The reaction mixture is stirred at room temperature for 15 hours and then stirred into water. The resulting mixture is extracted three times with ethyl acetate. The combined organic phases are dried over sodium sulfate and then concentrated under reduced pressure. This gives 4.28 mmol (86% of theory) of 3-cyano-5-chloro-6-(2-chloro-4-fluorophenyl)-7-(4-methylpiperidino)-pyrazolo[1,5-a)]pyrimidine.

logP (pH=2.3)=4.88

### Example 4

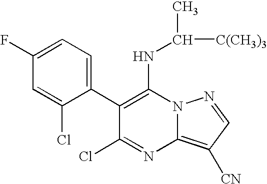

The compound of the formula given above is prepared by the method given in range 4.

HPLC: logP=4.92

### Example 5

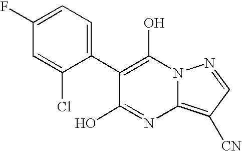 Process (f):

48 g (0.184 mol) of dimethyl 2-chloro-4-fluorophenylmalonate are mixed with 19.91 g (0.184 mol) of 4-cyano-5-amino-pyrazole and with 37.55 g (0.203 mol) of tri-n-butylamine, and the mixture is stirred at 180° C. for 6 hours. The methanol formed during the reaction is continuously distilled off. The reaction mixture is then cooled to room temperature. At 95° C. and 1 mbar, volatile components are distilled off. The residue obtained is 6-(2-chloro-4-fluorophenyl)-5,7-dihydroxypyrazolo[1,5-a]pyrimidine-3-carbonitrile in the form of a crude product which is used without additional purification for further synthesis.

### Example 6

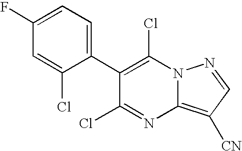 Process (e):

The crude 6-(2-chloro-4-fluorophenyl)-5,7-dihydroxypyrazolo[1,5-a]pyrimidine-3-carbonitrile obtained according to Example 6 is dissolved in 367.3 g (2.395 mol) of phosphorus oxychloride. At room temperature, 31.95 g (0.153 mol) of phosphorus pentachloride are added a little at a time. The mixture is then boiled under reflux for 12 hours. The volatile components are distilled off under reduced pressure, dichloromethane is added to the residue and the mixture is washed with water. The organic phase is dried over sodium sulfate and concentrated under reduced pressure. The residue is chromatographed on silica gel using 3 parts of cyclohexane and 1 part of ethyl acetate as mobile phase. This gives 21 g of 95.7% pure 3-cyano-5,7-dichloro-6-(2-chloro-4-fluorophenyl)pyrazolo[1,5-a]pyrimidine.

HPLC: logP=3.49

### Example A

Botrytis Test (Bean)/Protective

Solvents: 24.5 parts by weight of acetone

24.5 parts by weight of dimethylacetamide
   Emulsifier: 1.0 part by weight of alkylaryl polyglycol ether

To produce a suitable preparation of active compound, 1 part by weight of active compound is mixed with the stated amount of solvents and emulsifier, and the concentrate is diluted with water to the desired concentration.

To test for protective activity, young plants are sprayed with the preparation of active compound at the stated application rate. After the spray coating has dried on, 2 small pieces of agar colonized by Botrytis cinerea are placed onto each leaf. The inoculated plants are placed in a dark chamber at about 20° C. and 100% relative atmospheric humidity.

The size of the infected areas on the leaves is evaluated 2 days after the inoculation. 0% means an efficacy which corresponds to that of the control, whereas an efficacy of 100% means that no infection is observed.

In this test, the compound according to the invention listed in Example 1 showed, at an application rate of 500 g/ha, an efficacy of more than 90%.

1. A pyrazolopyrimidine of the formula 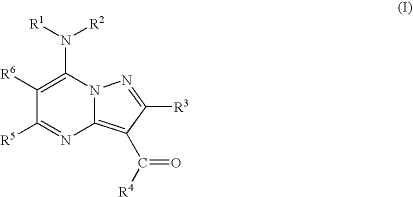
in which
R¹ represents optionally substituted alkyl, optionally substituted alkenyl, optionally substituted alkynyl, optionally substituted cycloalkyl or represents optionally substituted heterocyclyl,
R² represents hydrogen or alkyl, or
R¹ and R² together with the nitrogen atom to which they are attached represent an optionally substituted heterocyclic ring,
R³ represents hydrogen, halogen, optionally substituted alkyl or optionally substituted cycloalkyl,
R⁴ represents substituted alkyl, optionally substituted alkenyl, optionally substituted alkynyl, optionally substituted cycloalkyl or optionally substituted benzyl,
R⁵ represents halogen, optionally substituted alkyl, optionally substituted alkoxy, optionally substituted alkylthio, optionally substituted alkylsulfinyl or optionally substituted alkylsulfonyl and
R⁶ represents optionally substituted aryl.

2. The pyrazolopyrimidine of the formula (I) as claimed in claim 1
R¹ represents alkyl having 1 to 6 carbon atoms which may be mono- to penta-substituted by identical or different substituents from the group consisting of halogen, cyano, hydroxyl, alkoxy having 1 to 4 carbon atoms and cycloalkyl having 3 to 6 carbon atoms, or
R¹ represents alkenyl having 2 to 6 carbon atoms which may be mono- to trisubstituted by identical or different substituents from the group consisting of halogen, cyano, hydroxyl, alkoxy having 1 to 4 carbon atoms and cycloalkyl having 3 to 6 carbon atoms, or
R¹ represents alkynyl having 3 to 6 carbon atoms which may be mono- to trisubstituted by identical or different substituents from the group consisting of halogen, cyano, alkoxy having 1 to 4 carbon atoms and cycloalkyl having 3 to 6 carbon atoms, or
R¹ represents cycloalkyl having 3 to 6 carbon atoms which may be mono- to trisubstituted by identical or different substituents from the group consisting of halogen and alkyl having 1 to 4 carbon atoms, or
R¹ represents saturated or unsaturated heterocyclyl having 5 or 6 ring members and 1 to 3 heteroatoms, such as nitrogen, oxygen and/or sulfur, where the heterocyclyl may be mono- or disubstituted by halogen, alkyl having 1 to 4 carbon atoms, cyano, nitro and/or cycloalkyl having 3 to 6 carbon atoms,
R² represents hydrogen or alkyl having 1 to 4 carbon atoms, or
R¹ and R² together with the nitrogen atom to which they are attached represent a saturated or unsaturated heterocyclic ring having 3 to 6 ring members, where the heterocycle may contain a further nitrogen, oxygen or sulfur atom as ring member and where the heterocycle may be substituted up to three times by fluorine, chlorine, bromine, alkyl having 1 to 4 carbon atoms and/or haloalkyl having 1 to 4 carbon atoms and 1 to 9 fluorine and/or chlorine atoms,
R³ represents hydrogen, fluorine, chlorine, bromine, iodine, alkyl having 1 to 4 carbon atoms, haloalkyl having 1 to 4 carbon atoms and 1 to 9 halogen atoms or represents cycloalkyl having 3 to 6 carbon atoms,
R⁴ represents haloalkyl having 1 to 6 carbon atoms, alkenyl having 2 to 6 carbon atoms, alkynyl having 2 to 6 carbon atoms, cycloalkyl having 3 to 6 carbon atoms or represents benzyl,
R⁵ represents fluorine, chlorine, bromine, alkyl having 1 to 4 carbon atoms, alkoxy having 1 to 4 carbon atoms, alkylthio having 1 to 4 carbon atoms, alkylsulfinyl having 1 to 4 carbon atoms or alkylsulfonyl having 1 to 4 carbon atoms, and
R⁶ represents phenyl which may be mono- to tetrasubstituted by identical or different substituents from the group consisting of halogen, cyano, nitro, amino, hydroxyl, formyl, carboxyl, carbamoyl, thiocarbamoyl;
in each case straight-chain or branched alkyl, alkoxy, alkylthio, alkylsulfinyl or alkylsulfonyl having in each case 1 to 6 carbon atoms;
in each case straight-chain or branched haloalkyl, haloalkoxy, haloalkylthio, haloalkylsulfinyl or haloalkylsulfonyl having in each case 1 to 6 carbon atoms and 1 to 13 identical or different halogen atoms;
in each case straight-chain or branched haloalkenyl or haloalkenyloxy having in each case 2 to 6 carbon atoms and 1 to 11 identical or different halogen atoms;
in each case straight-chain or branched alkylamino, dialkylamino, alkylcarbonyl, alkylcarbonyloxy, alkoxycarbonyl, alkylsulfonyloxy, hydroximinoalkyl or alkoximinoalkyl having in each case 1 to 6 carbon atoms in the individual alkyl moieties;
cycloalkyl having 3 to 6 carbon atoms,
2,3-attached 1,3-propanediyl, 1,4-butanediyl, methylenedioxy (—O—CH₂—O—) or 1,2-ethylenedioxy (—O—CH₂—CH₂—O—), where these radicals may be mono- or polysubstituted by identical or different substituents from the group consisting of halogen, alkyl having 1 to 4 carbon atoms and haloalkyl having 1 to 4 carbon atoms and 1 to 9 identical or different halogen atoms.

3. The pyrazolopyrimidine of the formula (I) as claimed in claim 1 or 2, in which
R¹ represents a radical of the formula 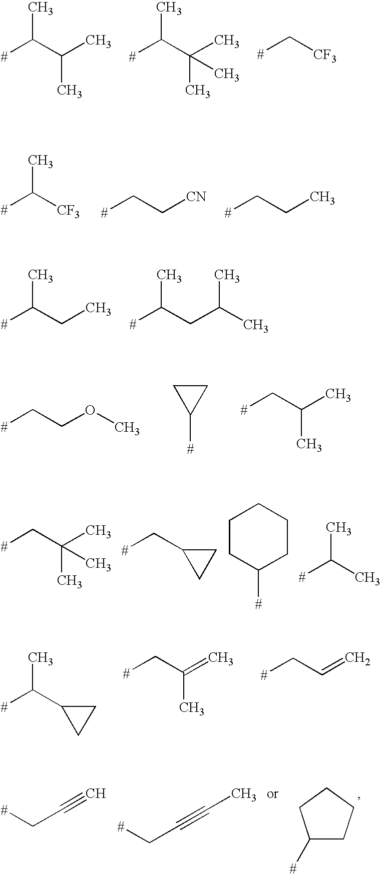
where # denotes the point of attachment and where, for the radicals which may be present in optically active form, each of the possible stereoisomers or mixtures thereof may be present,
R² represents hydrogen, methyl, ethyl or propyl, or
R¹ and R² together with the nitrogen atom to which they are attached represent pyrrolidinyl, piperidinyl, morpholinyl, thiomorpholinyl, piperazinyl, 3,6-dihydro-1(2H)-piperidinyl or tetrahydro-1(2H)-pyridazinyl, where these radicals may be substituted by 1 to 3 fluorine atoms, 1 to 3 methyl groups and/or trifluoromethyl,
or
R¹ and R² together with the nitrogen atom to which they are attached represent a radical of the formula 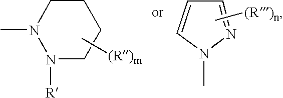
in which
R′ represents hydrogen or methyl,
R″ represents methyl, ethyl, fluorine, chlorine or trifluoromethyl,
m represents the number 0, 1, 2 or 3, where R″ represents identical or different radicals, if m represents 2 or 3,
R′″ represents methyl, ethyl, fluorine, chlorine or trifluoromethyl and
n represents the number 0, 1, 2 or 3, where R′″ represents identical or different radicals if n represents 2 or 3,
R³ represents hydrogen, fluorine, chlorine, bromine, iodine, methyl, ethyl, isopropyl, cyclopropyl, cyclobutyl, cyclopentyl, cyclohexyl, trifluoromethyl, 1-trifluoromethyl-2,2,2-trifluoroethyl or heptafluoroisopropyl,
R⁴ represents haloalkyl having 1 to 4 carbon atoms, alkenyl having 3 to 5 carbon atoms, alkynyl having 3 to 5 carbon atoms, cyclopropyl, cyclopentyl, cyclohexyl or represents benzyl,
R⁵ represents fluorine, chlorine, bromine, methyl, ethyl, methoxy, ethoxy, methylthio, methyl sulfinyl or methyl sulfonyl, and
R⁶ represents phenyl which may be mono- to trisubstituted by identical or different substituents from the group consisting of fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl; methoximinoethyl, ethoximinoethyl, cyclopropyl, cyclobutyl, cyclopentyl or cyclohexyl,
2,3-attached 1,3-propanediyl, methylenedioxy (—O—CH₂—O—) or 1,2-ethylenedioxy (—O—CH₂—CH₂—O—), where these radicals may be mono- or polysubstituted by identical or different substituents from the group consisting of fluorine, chlorine, methyl, ethyl, n-propyl, i-propyl and trifluoromethyl.

4. The pyrazolopyrimidine of the formula (I) as claimed in claims 1 or 2, in which
R⁴ represents CF₃, CCl₃, allyl, propargyl, cyclopropyl or benzyl,
R⁵ represents fluorine, chlorine, bromine, methyl, methoxy or methylthio and
R⁶ represents 2,4-, 2,5- or 2,6-disubstituted phenyl or 2-substituted phenyl or represents 2,4,6-trisubstituted phenyl, where the substituents are selected from the group consisting of fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl; methoximinoethyl, ethoximinoethyl, cyclopropyl, cyclobutyl, cyclopentyl, cyclohexyl,
2,3-attached 1,3-propanediyl, methylenedioxy (—O—CH₂—O—) and 1,2-ethylenedioxy (—O—CH₂—CH₂—O—), where these radicals may be mono- or polysubstituted by identical or different substituents from the group consisting of fluorine, chlorine, methyl, ethyl, n-propyl, i-propyl and/or trifluoromethyl.

5. A process for preparing pyrazolopyrimidines of the formula (I) according to claim 1, characterized in that
a) cyano compounds of the formula 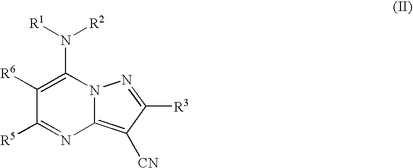
in which
R¹, R², R³, R⁵ and R⁶ are as defined above are reacted with Grignard compounds of the formula
R⁷—Mg—X (III)
in which
R⁷ represents substituted alkyl, optionally substituted alkenyl, optionally substituted alkynyl, optionally substituted cycloalkyl or optionally substituted benzyl and
X represents chlorine, bromine or iodine
in the presence of a catalyst and in the presence of a diluent,
or
b) pyrazolopyrimidines of the formula 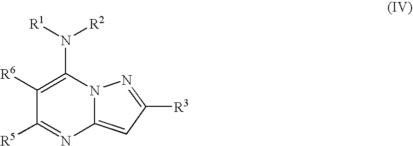
in which
R¹, R², R³, R⁵ and R⁶ are as defined above
are reacted with acid halides of the formula 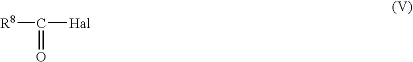
in which
R⁸ represents substituted alkyl, optionally substituted alkenyl, optionally substituted alkynyl, optionally substituted cycloalkyl or optionally substituted benzyl and
Hal represents chlorine or bromine,
or
with acid anhydrides of the formula 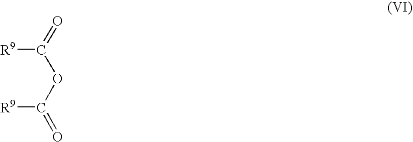
in which
R⁹ represents substituted alkyl or optionally substituted benzyl,
or other activated carboxylic acid derivatives, such as 4-dimethylaminopyridine acid anhydride adducts,
in each case in the presence of a catalyst and, if appropriate, in the presence of a diluent.

6. A composition for controlling unwanted microorganisms, characterized in that it comprises at least one pyrazolopyrimidine of the formula (I) as claimed in claims 1 or 2, in addition to extenders and/or surfactants.

7. The composition as claimed in claim 6, comprising at least one further fungicidally or insecticidally active compound.

8. (canceled)

9. A method for controlling unwanted microorganisms, characterized in that pyrazolopyrimidines of the formula (I) as claimed in claims 1 or 2 are applied to the unwanted microorganisms and/or their habitat.

10. A process for preparing compositions for controlling unwanted microorganisms, characterized in that pyrazolopyrimidines of the formula (I) as claimed in one or more of claims 1 or 2 are mixed with extenders and/or surfactants.

11. The pyrazolopyrimidine of the formula (I) as claimed in claim 3, in which
R⁴ represents CF₃, CCl₃, allyl, propargyl, cyclopropyl or benzyl,
R⁵ represents fluorine, chlorine, bromine, methyl, methoxy or methylthio and
R⁶ represents 2,4-, 2,5- or 2,6-disubstituted phenyl or 2-substituted phenyl or represents 2,4,6-trisubstituted phenyl, where the substituents are selected from the group consisting of fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl; methoximinoethyl, ethoximinoethyl, cyclopropyl, cyclobutyl, cyclopentyl, cyclohexyl,
2,3-attached 1,3-propanediyl, methylenedioxy (—O—CH₂—O—) and 1,2-ethylenedioxy (—O—CH₂—CH₂—O—), where these radicals may be mono- or polysubstituted by identical or different substituents from the group consisting of fluorine, chlorine, methyl, ethyl, n-propyl, i-propyl and/or trifluoromethyl.

12. A composition for controlling unwanted microorganisms, characterized in that it comprises at least one pyrazolopyrimidine of the formula (I) as claimed in claim 3, in addition to extenders and/or surfactants.

13. A composition for controlling unwanted microorganisms, characterized in that it comprises at least one pyrazolopyrimidine of the formula (I) as claimed in claim 4, in addition to extenders and/or surfactants.

14. A method for controlling unwanted microorganisms, characterized in that pyrazolopyrimidines of the formula (I) as claimed in claim 3 are applied to the unwanted microorganisms and/or their habitat.

15. A method for controlling unwanted microorganisms, characterized in that pyrazolopyrimidines of the formula (I) as claimed in claim 4 are applied to the unwanted microorganisms and/or their habitat.

16. A process for preparing compositions for controlling unwanted microorganisms, characterized in that pyrazolopyrimidines of the formula (I) as claimed in claim 3 are mixed with extenders and/or surfactants.

17. A process for preparing compositions for controlling unwanted microorganisms, characterized in that pyrazolopyrimidines of the formula (I) as claimed in claim 4 are mixed with extenders and/or surfactants.
